(19) United States
(12) Patent Application Publication
(10) Pub. No.: US 2017/0361124 A1
(43) Pub. Date: Dec. 21, 2017

(54) TREATMENT OF EYE CONDITION USING ADJUSTABLE LIGHT

(71) Applicant: SORAA, INC., FREMONT, CA (US)

(72) Inventors: JEFFERY ROBERT PARKER, PLEASANTON, CA (US); AURELIEN J.F. DAVID, SAN FRANCISCO, CA (US)

(21) Appl. No.: 15/383,897

(22) Filed: Dec. 19, 2016

Related U.S. Application Data
(60) Provisional application No. 62/352,779, filed on Jun. 21, 2016.

(51) Int. Cl. A61N 5/06

(52) U.S. Cl. CPC A61N 5/0613; A61N 2005/0662; A61N 2005/0661; A61N 2005/0626; A61N 2005/0654; A61N 2005/0635; A61N 2005/0659

(57) ABSTRACT

A method for treating an eye condition of a user using at least one light source, the at least one light source having an output which is adjustable in at least one of spectrum shape, intensity, or duration, the method comprising: (a) obtaining information relating to an eye condition of the user; (b) determining a dose of light to treat the eye condition, the dose including one or more of a particular spectrum shape, a particular intensity, or a particular duration; and (c) causing the at least one light source to adjust the output to deliver the dose.

### CROSS-REFERENCE TO RELATED APPLICATIONS

This application claims the benefit of U.S. Provisional Application No. 62/352,779, filed Jun. 21, 2016, the entire disclosure of which is incorporated herein by reference.

### FIELD OF DISCLOSURE

The present invention relates generally to treating eye conditions, and, more specifically, to treating eye conditions using light having an adjustable spectrum and/or intensity.

### BACKGROUND

As used herein, “eye condition” refers to a condition negatively affecting a user's eye sight. The type of condition may vary. For example, in one embodiment, the condition is a physical abnormality with the eye, such as a distorted shape, as is found in myopia and hyperopia. The information obtained in connection with these conditions may be very general, such as, a general diagnosis (e.g., myopia), or more specific, for example, a quantification of the eye deformity or the actual time spent outside in natural sunlight. Alternatively, the condition may be a propensity for an abnormality. Determining such propensities may be based on, such as, for example, hereditary information (e.g. a parent had myopia) or behavior or environmental information (e.g. the user spends an inordinate amount of time indoors or has a deficient exposure to natural light). The term “treat” or “treating” in the context of an eye condition refers to remediating, preventing, or mitigating an eye condition.

Eye conditions, such as myopia and hyperopia, have been managed traditionally through corrective eye glasses/contact lenses, and, more recently, through surgery. Although these conventional approaches tend to treat the eye condition, they have significant shortcomings. For example, prescription glasses tend to be cumbersome and expensive, especially over time and for growing children who need new prescription glasses frequently. On the other hand, surgery is invasive, prone to complications (infection), and is expensive to the point that only a small segment of the population can afford this approach. Moreover, the operation is available only for adults, and only for certain eye conditions. For many, particularly those in impoverished or developing communities, surgery is not an option. Therefore, there is a need for a convenient, non-invasive, and inexpensive approach to correct eye conditions. The present invention fulfills this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Applicants recognize that a user's eye condition may be improved by a light source having an adjustable spectrum and/or intensity. Specifically, recent studies have shown that various eyesight conditions, such as myopia and hyperopia, are related to inadequate light exposure, both in humans and animals. Applicants recognize that, if eye conditions can be caused by deficiencies in exposure to certain wavelengths of light or to adequate light intensity, then these eye conditions can be improved by increased exposure to certain wavelengths or exposure to higher light intensities. In one embodiment, the invention involves a system, which assesses the state of a user's vision, and delivers a tailored light to correct eye conditions or potential conditions, the tailored light may be dynamically adapted to the user's condition as it evolves. Embodiments of the invention address these needs by generating a modified light to treat eye conditions, while retaining a high quality of light.

In one embodiment, the invention involves a method for correcting an eye condition of a user using at least one light source, the at least one light source having an output which is adjustable in at least one of spectrum shape or intensity, the method comprising: (a) obtaining information relating to an eye condition of the user; (b) determining a dose of light to treat the eye condition, the dose including one or more of a particular spectrum shape, a particular intensity, or a particular duration; and (c) causing the at least one light source to adjust the output to deliver the dose.

In one embodiment, the invention involves a system for treating an eye condition of a user, comprising: a digital processor operatively connected to at least one light source; an input and output interface operatively connected to the digital processor for receiving and outing instructions and data to and from the digital processor; memory operatively connected to the digital processor, the memory being configured with instructions for causing the processor to execute the following steps: (a) obtaining information relating to the eye condition of the user; (b) determining a dose of light to treat the eye condition, the dose including one or more of a particular spectrum shape, a particular intensity, or a particular duration; and (c) causing the at least one light source to adjust the output to deliver the dose.

### BRIEF DESCRIPTION OF FIGURES

FIG. 1 is one embodiment of the system of the present invention.

FIG. 2(a)-FIG. 2(d) show spectrum embodiments of the present invention spectral content at various wavelenths.

FIG. 3 shows another spectrum embodiment having a lack of radiation in a specific wavelength range, and a presence of radiation in another wavelength range.

FIG. 4 shows the absolute wall-plug efficiency (WPE) of III-Nitride LEDs in the wavelength range 390-440 nm at a current density of 100 A·cm-2.

FIG. 5 shows the absolute external quantum efficiency (EQE) of III-Nitride LEDs in the wavelength range 390-440 nm, at a current density of 100 A·cm.

FIG. 6 shows the absolute WPE of III-Nitride LEDs in the wavelength range 390-440 nm, at various current densities in the range 4-30 A·cm-2.

FIG. 7 shows SPDs at various CCTs, in absolute radiometric units of mW/nm/m².

### DETAILED DESCRIPTION

One embodiment of the present invention is a method for correcting an eye condition of a user using at least one light source, the at least one light source having an output which is adjustable in at least one of spectrum shape or intensity. In one embodiment, the method comprises: (a) obtaining information relating to an eye condition of the user's eye sight; (b) determining a dose of light to correct the eye condition, the dose including one or more of a particular spectrum shape, a particular intensity, and a particular duration; and (c) causing the at least one light source to adjust the output to deliver the dose.

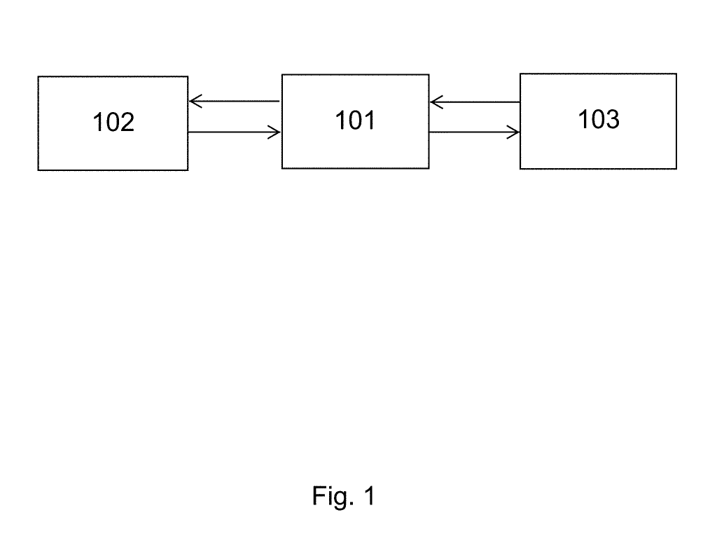

Referring to FIG. 1, one embodiment of the system 100 of present invention for correcting an eye condition is shown. The system 100 comprises a digital processor 101; a user interface 103 operatively connected to the digital processor for receiving and transmitting instructions and data to and from the digital processor; and memory 102 operatively connected to the digital processor. The memory is configured with instructions for causing the processor to execute the following steps: (a) obtaining information relating to the eye condition of the user; (b) determining a dose of light to treat the eye condition, the dose including one or more of a particular spectrum shape, a particular intensity, and a particular duration; and (c) causing at least one light source 105 to adjust the output to deliver the dose.

These elements are considered below in greater detail and with respect to selected, alternative embodiments.

In step (a), information is obtained relating to the eye condition of the user's eye. This information may include, for example, a diagnosis such as myopia or hyperopia, and/or measurements of the user's eye deformity. Alternatively, the information may relate to environmental factors that are known to lead to eye problems, such as a deficiency of natural sunlight. In still another embodiment, the information may relate to a propensity for an abnormality, and include, for example, hereditary information (e.g. a parent had myopia).

This information can be obtained in a variety of different ways. For example, in one embodiment, the eye condition information may be obtained by a third party, for example, an optometrist, other eye-care provider, or geneticist. The third party may perform the diagnostic, measurement, or file review of the user using conventional techniques and instruments, and then provide the eye condition information to the system. The information may be entered into the system manually, or it may be received as an electronic file from, for example, the “Cloud” 108 or other network.

Alternatively, the eye condition information may be obtained indirectly by monitoring the light exposure a user receives and correlating that exposure to an eye condition. The correlation may be based on established data of light exposure and eye conditions as discussed above. Additionally, additional correlations may be established over time using the present system as users are monitored with respect to both light exposure and eye conditions. Monitoring the user's light exposure can be performed using a conventional light monitor 107. For example, in one embodiment, the user may carry or wear a device for monitoring and recording that light to which he or she is exposed. This device may also be configured to quantify the intensity and/or wavelengths of the light received. This data may stored and periodically transmitted to the processor 101 using known wireless and cable communication links. It should be understood that the monitoring device 106 may be used also to monitor the light exposure of the user 110 for feedback purposes, discussed below.

In yet another embodiment, the system comprises a test module 107 or subsystem for obtaining eye condition information. For example, in one embodiment, the test system assesses the visual acuity of the user to determine existing eye conditions and screens for potential eye conditions. In some embodiments the test system detects preliminary symptoms of a condition that has not yet fully developed, even though the user's visual acuity is not yet affected, the system offers a diagnosis of the onset of a condition.

The test system can be, for example, a conventional visual acuity test, similar to that used by opticians. The test system may also be a lighter-footprint system for mobile use or use at home. The test system may be hardware or software-based. The test system may be embodied in a computer or IT system—in particular, the test may be on a smartphone, tablet, or laptop. For example, in one embodiment, test system is an application for smartphone or tablet. The application displays visual patterns on the screen, which elicit an eye response from the user. The eye response is tracked by the front camera of the smartphone or tablet using known static or dynamic test patterns. In various embodiments the test is self-administered, without requiring the presence of a health care professional. Such applications are well known and are commercially available for smartphones and tablet computers.

In some embodiments, the visual acuity test is repeated over time. For example, the test may be repeated every week, every month, every year. Repetitions over short period of time can help improve the accuracy of the diagnosis. Repetitions over longer period of time can help observe an evolution in the diagnosis.

It should be understood that testing and dosage steps can be done in an iterative fashion. Meaning that the invention contemplates repeatedly reevaluating the user's eye condition (in step (a)) and possibly recalculating the dose (in step (b)) and thus causing the light source to further adjust its light output to deliver the revised dose (in step (c)). The frequency of these iterations can be optimized by one of skill in the art in light of this disclosure. Such optimization may depend on a number of factors, including, the ease at which the eye condition can be determined, the expected time for an eye condition to change, advice from a health specialist.

In step (b), once the eye condition information is obtained, the light dose is determined to correct the eye condition. As used herein, the term “light dosage” or “light dose” or just “dose” refers to a quantification of light to be administered to a user. The light may be quantified in different ways including, for example, (a) a particular spectrum shape, (b) a particular intensity (e.g. illuminance (lux), including retinal illuminance (lux at eye), power density, etc.), or (c) a particular duration, or a combination thereof. In one embodiment, the dose includes information related to all three characteristics.

Research has shown spectrum shape, intensity, and/or duration of light can prevent/remediate eye conditions. Specifically, recent studies have shown that various eyesight conditions, such as myopia and hyperopia, are related to inadequate light exposure, both in humans and animals. For example, [Jones07] and [Rose08] identify the link between myopia and lack of exposure to high intensity light, such as that found in natural light (e.g., 10,000-100,000 lux), in contrast to typical indoor lighting environments (100 to 1,000 lux). [Jones07, Rose08] show that low levels of outdoor activity increase children's odds to develop myopia. Various articles show similar effects in animals. For example, [Lan14] shows that bright light can suppress myopia in chicken, and that intermittent episodes of bright light are more effective than continuous episodes.

Likewise, research has demonstrated specific effects from various wavelengths of light. For example, [Foulds13] has shown that rearing chicks in red light (peaking at 641 nm) causes progressive myopia, while rearing them in blue light (peaking at 440 nm) causes progressive hyperopia. Likewise, these effects can be reversed by light therapy. For example, as described in [Foulds13], exposure of chicks to a blue spectrum for a period of 21 days (the light being on for 12 hours each day) has been shown to reverse a myopia of −2.21 diopters to a hyperopia of +2.50 diopters.

These results are not limited to light having a specific color (or wavelength range), but also apply to broadband sources. [Czepita04] showed that fluorescent lighting is associated with a prevalence of hyperopia and astigmatism compared to incandescent lighting. This may be associated with the relatively larger amount of long-wavelength radiation in incandescent spectra, and the relatively larger amount of short-wavelength radiation in fluorescent spectra.

Further, these results are not limited to the visible range of radiation. Due to the beneficial effect of sunlight on the occurrence of eye conditions, it is believed that non-visible parts of the radiation spectrum may also play a role on eye development. For instance, Stephen B. Prepas, “Light, Literacy and the Absence of Ultraviolet Radiation in the Development of Myopia,” Medical Hypotheses (2008) 70, 635-637 proposes that ultra-violet radiation in the range 200-400 nm may reduce the development of myopia. Likewise, the spectrum of sunlight comprises a substantial amount of infrared radiation in the range 700-2500 nm or, more particularly, in the range 800-1300 nm.

In addition to the spectrum shape, it has also been found that light intensity can also contribute to eye abnormalities. The intensity may be expressed in lux, in watts per square meters or in other optical units, it may be measured at a given point in an illuminated room, or it may be a retinal intensity. For example, studies have shown that exposure to natural light tends to promote healthy eye development. Natural light has an intensity, which can reach or exceed 100,000 lux. One the other hand, indoor lighting has an intensity of about 100 lux to 500 lux. Thus, a person spending little time outside would receive a mere fraction of the light intensity someone exposed to natural light typically experiences. This lack of light intensity has been shown to lead to eye abnormalities such as myopia ([Jones07, Rose08]). Therefore, in another embodiment, the dose involves an increase in intensity in the light. For example, rather than a typical indoor light intensity of 100 lux to 500 lux, the dose of the present invention may range from 1,000 to 10,000 lux or even more depending upon the light source to treat the eye conditions as discussed above.

The influence of daylight likely involves both the high illuminance and high CCT (in other words, the relatively high amount of short-wavelength light) of daylight.

Therefore, a dose of light can be used to correct, improve or avoid an eye condition. The dose of light may vary depending on the needs of the user. For example, in some embodiments, preferential exposure to light having a wavelength of 300-500 nm counteracts myopia. Specifically, a dose may be light having a relatively high blue spectrum in which the blue spectrum is narrow-band blue light form a blue-emitting LED, for a period of time, for example two hours, at a desired illuminance, for instance 1,000 lux. Other examples of a blue-rich spectrum include a broadband spectrum with relatively high CCT (for instance 5000K, 6000K or above). Alternatively, in some embodiments, preferential exposure to light having a wavelength of 580-2500 nm may be used to counteract hyperopia.

In some embodiments, a schedule of exposure to the emitted spectrum is provided to the user. For example, the user is instructed to have at least one hour of exposure each day to the emitted spectrum. It should be understood that the entire dose may include one or more of the spectrums shape, intensity and duration. For example, in one embodiment, the dose is defined as just a particular spectrum or intensity. In another embodiment, the dose may be specified as a particular spectrum for a particular duration, or, alternatively, a particular intensity over, or a particular duration. Whether the dose includes duration, depends somewhat, on whether or not the user can be monitored for his or her exposure to light (as mentioned above) to determine the amount of light the user is receiving.

In one embodiment, the dose takes into consideration the user's location and the typical natural light received in that location as well as the user's behavior and exposure to the natural light. For example, the one embodiment, the system may determine the dose based upon the user's latitude and time of year to determine the likelihood of receiving natural light. Additionally, the system may consider how often the user is outdoors.

In some embodiments, exposure to the treatment is configured to correct a predetermined diopter rating in a predetermined amount of time. For instance, the dose (including illuminance, spectrum of the light source and light treatment schedule) is configured to modify the patient's vision by plus or minus one diopter in about one week, one month, three months, six months, one year, two years.

Determination of the dose may be undertaken in various ways. For example, in one embodiment, once the eye condition is determined as discussed in step (a) above, an objective is identified, which may include, for example, a desired eye shape and a time table (i.e., duration) for achieving/maintaining the objective. For instance, the eye shape objective may be to maintain the eye shape/elongation to within a predetermined value—e.g., less than 0.01 mm or 0.1 mm or 0.5 mm or 1 mm or 2 mm or 3 mm for a certain time (e.g. one year), or the eye shape objective may be a diopter correction required to correct an eye condition (less than 0.1 or 0.2 or 0.5 or 1 or 1.5 or 2, positive or negative) within a certain time (e.g., one year). Next, an algorithm determines the dose as a function of one or more parameters, which may include, for example, eye shape distortion, eye shape objective, treatment duration, light intensity, the shape of the SPD (including peaks in the spectrum, radiation or absence of radiation in a specific wavelength range), relative or absolute power density in various spectral ranges, CCT, and the user's schedule, just to name a few. The following is a simplistic algorithm for determining dosage:

Input D, eye shape distortion (diopters)

Input O, eye shape objective (diopters)

Input T, treatment duration (weeks)

Dosage: if D<0, then 300-400 nm at 1000 lux [(O−D)*3500/C] min/day.
If D>0, then 700-900 nm at 1000 lux [(O−D)*3500/C] min/day.
   Therefore, if, for example, a user has an eye shape distortion of −2.5 diopters, and has an eye shape objective of 0 diopters, and wants to correct the eye distortion within one year, then the following would be the dosage:

300-400 nm at 1000 lux [(0−(−2.5))*3500/52] min/day, or

300-400 nm at 1000 lux [169] min/day.

Obviously, this algorithm is simplistic, and one of skill in the art will understand that it may need to be modified to optimize the dosage as more data and studies become available. For example, the algorithm can be readily modified such that the dosage varies the intensity of the light (rather than the daily exposure) as a function of the treatment duration and the difference between the eye shape distortion and eye shape objective. In yet another embodiment, both the intensity of the light and the daily exposure vary as a function of the treatment duration and the difference between the eye shape distortion and eye shape objective. Still other variations and additional parameters will be obvious to one of skill in the art in light of this disclosure.

In Step (c), the light source 105 is adjusted to output light to deliver the dose to the user. To this end, the light source has an adjustable light control 104, which can be adjusted to vary the light's spectrum shape, its intensity, its duration, or a combination of two or more of these.

A light source configured to vary the spectrum may have different embodiments. Generally, although not necessarily, the light source is configured to favor certain wavelengths over others. For example, the light source may favor, for example, light in the violet or blue spectrum (i.e. wavelengths 400-430 nm and 430-470 nm, respectively), or light in the red spectrum (i.e. wavelengths 600-700 nm or 580-700 nm), or light in the ultra-violet range or infrared range. In connection with ultra-violet light (i.e., wavelengths 300-400 nm), there may be concern that deep ultra-violet may have other harmful effects. Therefore, the near-UV range may be preferable (e.g., the spectrum may have radiation in the range 360-400 nm or 380-400 nm, or have an emission peak at 360 nm, 365 nm, 370 nm, 375 nm, 380 nm, 385 nm, 390 nm, 395 nm, 400 nm). For the infrared range, radiation may be in the range 700-2000 nm or 800-1300 nm. It may be a broad emission or have a peak at a specific infrared wavelength.

One advantage of using non-visible wavelengths is that they may be added in substantial amounts without affecting the visual experience of the subject; for instance, the invention may emit a regular-looking spectrum with a CCT of 3000 K, but also having an intense ultra violet and/or infrared emission which do not modify the visual perception of the light but provide therapeutic benefits; or the invention may emit only non-visible radiation to provide therapeutic benefits.

In general, there is wide latitude in tuning an emitted spectrum. The spectrum may be characterized by various aspects of quality of light; including its absolute light level, and color rendition metrics such as correlated color temperature (CCT), distance from Planckian (Duv), color rendering index (CRI Ra, R9, IES TM-30 Rf, Rg) and others. Further, the spectrum may be characterized by other aspects which impact visual conditions, for example the ratio of red light to blue light, the absolute irradiance of blue light, the absolute irradiance of red light, the presence of infrared radiation, and others.

The emitted spectrum may be selected by imposing various constraints on these parameters. For example, the constraints may be that the emitted spectrum has a CCT of 3000K, a color rendering Ra of about 80, and a red to blue ratio suitable for treating the condition diagnosed by the test system. For instance, the red/blue ratio may be about 100:1, 50:1, 20:1, 10:1, 5:1, 2:1, 1:2, 1:5, 1:10, 1:20, 1:50, 1:100. These ratios may be computed by integrating the SPD in a blue range (such as 400-500 nm, 400-450 nm, 450-500 nm, 400-420 nm, 420-440 nm, 440-460 nm, 460-480 nm, 480-500 nm) and a red range (such as 600-700 nm, 575-625 nm, 600-650 nm, 625-675 nm, 650-700 nm, 600-620 nm, 620-640 nm, 640-660 nm, 660-680 nm) Such light has characteristics suitable for general indoor lighting applications, while also providing a beneficial effect for the visual condition of the user. Other CCTs are possible (such as warmer white with a CCT of 2000 to 2700K, or cooler white with a CCT of 4.000 to 10,000K). More generally, it is possible to impose constraints regarding the quality of the emitted light, together with constraints regarding its therapeutic aspects. In some embodiments of the invention, the spectrum is optimized against these various constraints; for example, the spectrum's CRI Ra is maximized, while maintaining a given ratio of blue light to red light. Standard numerical techniques can be employed to achieve this optimization. See, for example, U.S. patent application Ser. No. 14/531,545 and U.S. Pat. No. 9,368,695, hereby incorporated by reference.

In some embodiments, the emitted spectrum is substantially white light (i.e., it is on the Planckian locus or close to it), but lacks light in a given wavelength range. For example, it is possible to create white light by mixing violet, green-yellow and red light but with very little blue and cyan emission, which may be useful for treating some visual conditions. Likewise, it is generally possible to remove light in another spectral band while achieving white balance, and good color rendering (for example, CRI Ra above 80 or 90, or CRI R9 above 50 or 80 or 90). In some embodiments, the spectrum is substantially similar to daylight—for example to a reference illuminant such as D50, D65 or other, or an indoor daylight illuminant. In some embodiments the spectrum includes ultra-violet radiation. In some embodiments the spectrum includes infra-red radiation.

By preferentially outputting one or more wavelengths over other wavelengths, the spectrum is shaped. It should be understood, that various spectrum shapes could be used, for example, in one embodiment, a particular wavelength is amplified, appearing as a spike in the spectrum. In another embodiment, a range of wavelengths is amplified relative to the other wavelengths to create a bump in the spectrum shape. In yet another embodiment, different wavelengths may be selectively amplified to, for example, taper upwardly or downwardly with respect to the other wavelengths. Still other embodiments of spectrum shape will be obvious to those of skill in the art in light of this disclosure.

Alternatively, rather than amplifying one wavelength over another, a spectrum may be shaped by reducing non-preferred wavelength. For example, filters (either absorbing or reflective) can be placed in the path of emitted light to remove light in a determined wavelength range. Techniques for creating a spectrum having a white chromaticity and a lack of light in a wavelength range can be found in U.S. patent application Ser. No. 14/531,545 and U.S. Pat. No. 9,368,695.

Rather than or in addition to adjusting the light source to shape the spectrum, the light source may also be configured to emit light at varying intensity. Such configurations are well known and, thus, the details of which are not disclosed herein. In some embodiments, the illuminance is higher than that found in conventional lighting application, and may be similar to the illuminance of daylight. For example the illuminance may be at least 1,000 lux, 10,000 lux, or even 100,000 lux. Such high lux levels may for instance be achieved by a directional light source, which can direct light towards the field of view of the user. The illuminance may be at a surface in a location, or a retinal illuminance.

The light source 105 may comprise a variety of light-emitting systems. This may include light bulbs and lamps, lighting fixtures and luminaires. This may also include display systems (including screens). This may further include a dedicated system for sending light preferentially in the user's eyes. For instance, the light source may be a mask or a pair of goggles/glasses mounted on the user's head, which direct radiation toward his eyes. The light source may be a dedicated directional light source having light emission directed towards the user's eye, or more preferentially towards a part of the user's eye. Light delivery may be highly directional. For instance the light source may be collimated from an LED or a laser diode and be directed towards the user's face, eye or pupil. In some cases, a laser-based light source tracks the user's movements and sends light in his eye, compensating for his movements. In some cases, the radiation is invisible (i.e. UV, near UV or infrared) and therapy is administered without impacting the visual function of the user. In some cases, only a specific area of the user's eye is illuminated. For instance, only a specific area of the retina (for instance phovea/paraphovea/periphery/top/bottom/etc.) receives light.

In yet another embodiment, the system 100 in FIG. 1 is integrated with the light source, and thus uses internal communications to alter the light source's output. For example, the light source 105 may be one or more lamps for backlighting a display screen of a tablet, computer or television. The backlighting may be adjusted to deliver the dose to the user as determined above. Still other embodiments and light configurations will be understood by those of skill in the art in light of this disclosure.

Causing the light source 105 to alter the output to deliver the dose can be achieved in a variety of ways. For example, in one embodiment, the system wirelessly communicates (e.g., using WiFi, Bluetooth or near field communication) to the light control 104 to alter the output of the light source 105 to deliver the dose. For example, in one embodiment, the light source is a desk lamp or other light fixture to which the user is exposed on a frequent daily basis. In this embodiment, the system may wirelessly communicate with the lamp or light fixture and thus cause it to alter is output as described above. In another embodiment, the system is hardwired to the light control 104 to cause it to alter the light source 105 and deliver the dose. For example, in one embodiment, the light control 104 is networked to the processor 101 through the powerline using known powerline communication protocols. In yet embodiment, the system is connected to the light source using TRC power light communication technology, as is known in the art.

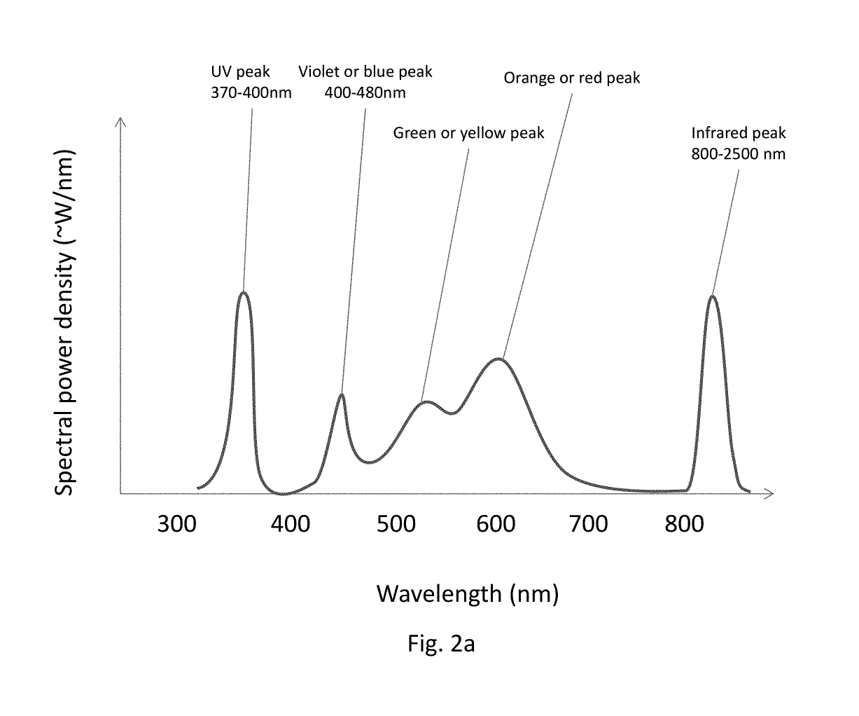

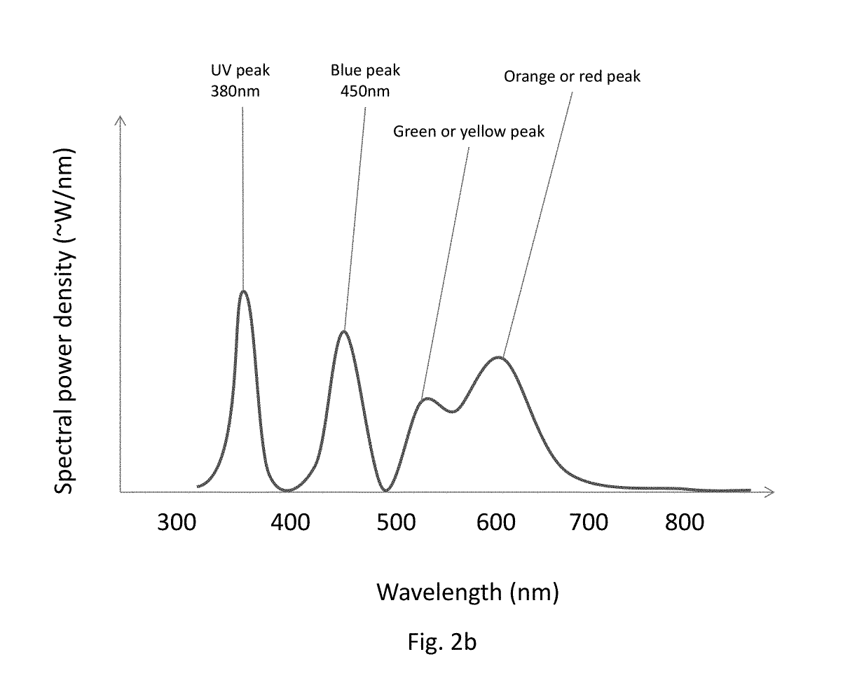

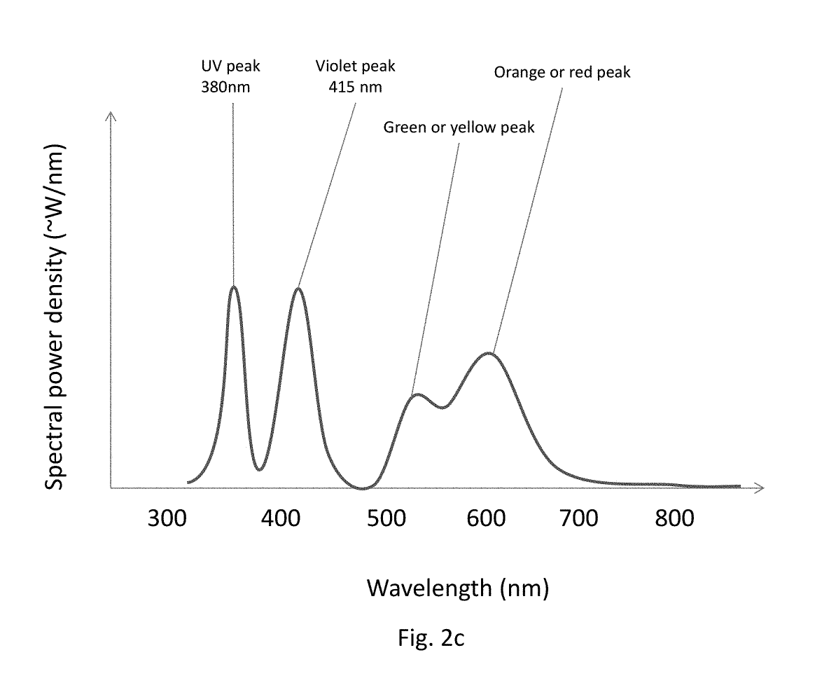

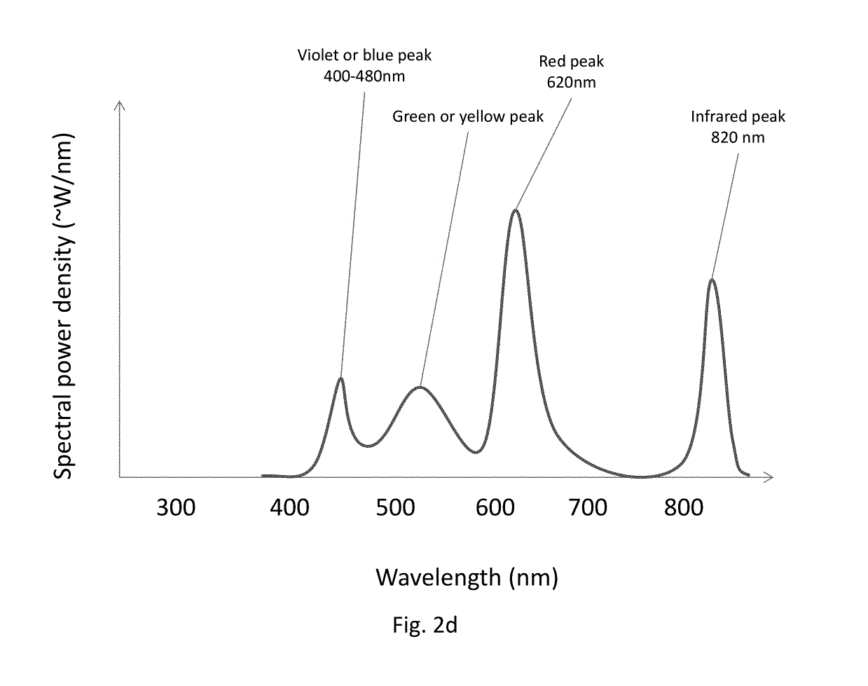

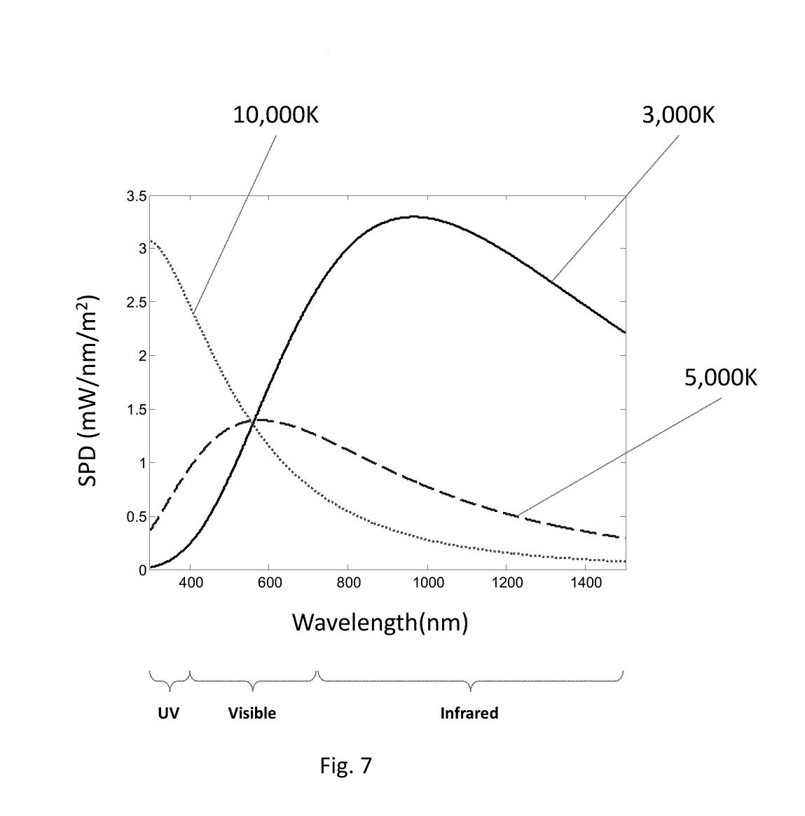

Referring to FIGS. 2(a)-(c), 3, and 7, examples of various spectrums used in connection with the present invention are shown. FIG. 7 shows SPDs at various CCTs, in absolute radiometric units of mW/nm/m². All these SPDs are normalized to have the same illuminance of 100 lux. Their respective CCTs are 3000K, 5000K, 10000K. FIG. 7 is indicative of the order of magnitude for optical power densities, which may be employed in embodiments where an illuminance of 100 lux is used. Particular examples are discussed below in connection with FIG. 7.

In the case where a high dose of long-wavelength visible radiation is sought, a warm-white SPD (such as 3,000K) may be used. This may correspond to a spectral flux density of about 0.5 or 1 or 1.5 or 2 mW/nm/m² at a wavelength in the range 500-700 nm, or a spectrally-integrated flux density of about 50 or 100 or 150 or 200 mW/m² (or within a range 50-200 mW/m²) integrated over a 100 nm wavelength range (for instance, 500-600 nm or 550-650 nm or 600-700 nm).

In the case where a high dose of infrared radiation is sought, a warm-white SPD (such as 3000K) may be used. This may correspond to a spectral power density of about 2 or 3 mW/nm/m² at a wavelength in the range 800-1500 nm, or a spectrally-integrated flux density of about 200 or 300 mW/m² (or within a range 200-300 mW/m²) over a 100 nm wavelength range (for instance, 800-900 nm or 900-1000 nm or 1000-1100 nm or 1100-1200 nm or 1200-1300 nm or 1300-1400 nm or 1400-1500 nm).

In the case where a high dose of short-wavelength visible radiation is sought, a cool-white SPD (such as 5,000K or 10,000K) may be used. This may correspond to a spectral flux density of about 0.5 or 1 or 1.5 or 2 mW/nm/m² at a wavelength in the range 400-500 nm, or a spectrally-integrated flux density of about 5 or 10 or 15 or 20 mW/m² (or within a range 5-20 mW/m²) integrated over a 10 nm wavelength range (for instance, 400-410 nm or 410-420 nm or 420-430 nm or 430-440 nm or 440-450 nm or 450-460 nm or 460-470 nm or 470-480 nm or 480-490 nm or 490-500 nm), or a spectrally-integrated flux density of about 25 or 50 or 75 or 100 mW/m² (or within a range 25-100 mW/m²) integrated over a 50 nm wavelength range (for instance, 400-450 nm or 425-475 nm or 450-500 nm).

In the case where a high dose of UV radiation is sought, a cool-white SPD (such as 5,000K or 10,000K) may be used. This may correspond to a spectral flux density of about 0.5 or 1 or 2 or 3 mW/nm/m² at a wavelength in the range 300-400 nm, or a spectrally-integrated flux density of about 5 or 10 or 20 or 30 mW/m² (or within a range 5-30 mW/m²) integrated over a 10 nm wavelength range (for instance, 300-310 nm or 310-320 nm or 320-330 nm or 330-340 nm or 340-350 nm or 350-360 nm or 360-370 nm or 370-380 nm or 380-390 nm or 390-400 nm).

If a different illuminance is desired, all these values can be scaled. For instance, embodiments may have an illuminance of 500 lux, 1000 lux, 5000 lux—in these cases, the above power density values are respectively multiplied by 5×, 10× 50× and so on. In the case where a retinal illuminance is sought, the aforementioned power densities are also retinal.

Furthermore, although the previous examples look at blackbody SPDs, the CCT does not have to be directly tied to the dose. For instance, any SPD may contain a high dose of UV or infrared light (since these are invisible or nearly-invisible to our vision system).

FIG. 2a illustrates one embodiment of the spectral power distribution of the present invention, with wavelength ranges in both the shorter wavelength range (i.e., 300-400 nm for counteracting myopia), and the longer wavelength range (i.e., 800-2500 nm for counteracting hyperopia). Specifically, referring to the figure, the SPD comprises a UV peak, a violet or blue peak, a yellow-green peak, an orange-red peak, and infrared peak. In some embodiments, at least 1% (or 5%, or 10%, or 20%) of the total power of the SPD is in the ultra-violet range (for instance, the range 300-400 nm). In some embodiments, at least 1% (or 5%, or 10%, or 20%) of the total power of the SPD is in the infrared range (for instance, the range 800-2500 nm). In such calculations, the total power is integrated in a wide enough range such that all radiation is included—for instance, the range 300-2500 nm. This would provide a surrogate spectrum for natural light. Thus, in this embodiment, the same spectrum may be used to treat either myopia or hyperopia, or more generally an eye condition which is caused by the absence of UV and infrared light in standard LED light sources.

In such embodiments, comparison to natural light can be made by defining a standard illuminant (Blackbody or daylight) having the same CCT as the SPD of the embodiment, and the same illuminance. In some embodiments, the total power of the SPD in the UV range (for instance, in the range 300-400 nm) is at least 5% (or 10%, 20%, 50%, 100%) of the total power in the UV range of the reference illuminant. In some embodiments, the total power of the SPD in the UV range is within 10%-50% of the total power in the UV range of the reference illuminant. In some embodiments, the total power of the SPD in the infrared range (for instance, in the range 800-2500 nm) is at least 5% (or 10%, 20%, 50%, 100%) of the total power in the infrared range of the reference illuminant. In some embodiments, the total power of the SPD in the infrared range is within 10%-50% of the total power in the infrared range of the reference illuminant.

Alternatively, a spectrum may be used which emphasizes the shorter or higher wavelengths. For example, referring to FIG. 2(b), the spectrum emphasize shorter wavelength light as compared with longer wavelength light. In FIG. 2 (b), the spectrum has a pronounced blue peak at 450 nm, little cyan and violet light, and a pronounced UV peak at 380 nm. This is suited for embodiments in which therapeutic effects are expected from blue and UV light.

Referring to FIG. 2(c), the spectrum emphasize shorter wavelength light as compared with longer wavelength light. In FIG. 2 (c), the spectrum has a pronounced violet peak at 415 nm, little cyan and blue light, and a pronounced UV peak at 380 nm. This is suited for embodiments where therapeutic effects are expected from violet and UV light.

Other variations may emphasize any combination of UV, violet, blue and cyan light.

FIG. 2(d) shows the alternative in which the spectrum emphasizes longer wavelength light as compared to shorter wavelength light. The spectrum has a pronounced red peak at 620 nm and a pronounced infrared peak at 820 nm.

Other embodiments may emphasize any combination of orange, infrared and red light.

UV, violet, blue, cyan, green, yellow, orange, red and infrared emission may come from solid-state emitters (including lasers and laser diodes) and/or from wavelength converters (including phosphors and quantum dots). The type of emitter determines the sharpness of the emission peaks—for instance lasers are very narrow, LEDs are relatively narrow and phosphors are relatively broad. Therefore the choice of emitter may be dictated by the need to include a large amount of radiation in a specific spectral range.

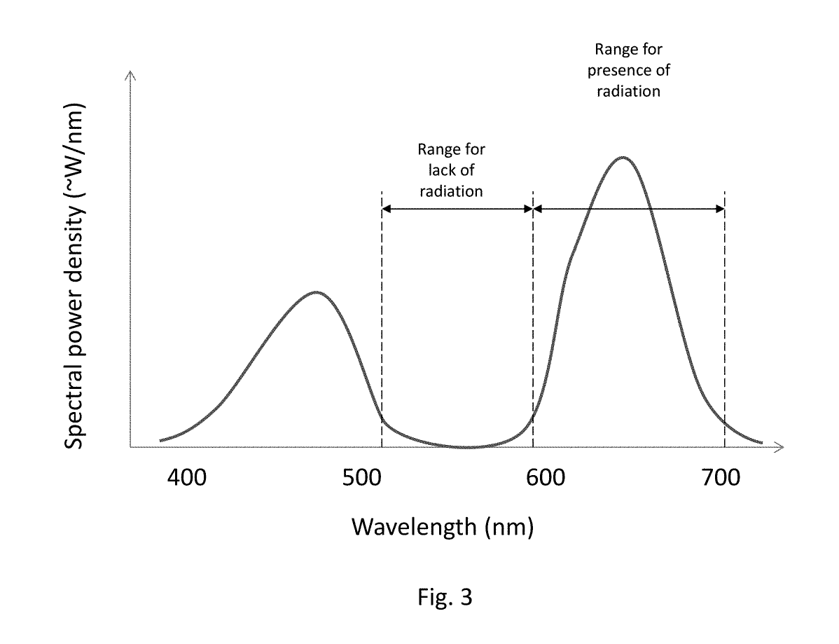

FIG. 3 illustrates another embodiment having a lack of radiation in a specific wavelength range (e.g., 500-600 nm), and a presence of radiation in another wavelength range (e.g., 600-700 nm). The lack of radiation may be characterized by how much radiation is included in the range. For instance, the SPD may be characterized in that less than 0.1% (or 1%, 2%, 5%, 7%, 10%, 15%, 20%) of its total power is contained within a predetermined range. Embodiments of the invention include a method comprising of 1) determining a wavelength range (for instance, based on step (a) above), and 2) configuring a light emitter such that its SPD contains less than a predetermined fraction of its total power within the wavelength range.

Conversely, embodiments include SPDs having a sufficient presence/amount of radiation within a wavelength range, as also shown on FIG. 3. For instance, the SPD may be characterized in that at least 0.1% (or 1%, 2%, 5%, 10%, 15%, 20%, 25%, 30%, 40%, 50%, 60%, 70%, 80%) of its total power is contained within a predetermined range. Embodiments of the invention include a method comprising of 1) determining a wavelength range (for instance, based on step (a) above), and 2) configuring a light emitter such that its SPD contains at least a predetermined fraction of its total power within the wavelength range.

Useful wavelength ranges for prescribing an amount of radiation include: 200-2500 nm, 300-1300 nm, 380-780 nm, 300-400 nm, 350-400 nm, 370-400 nm, 380-400 nm, 390-400 nm, 380-410 nm, 400-430 nm, 410-430 nm, 400-450 nm, 400-490 nm, 400-500 nm, 430-500 nm, 500-600 nm, 500-550 nm, 550-600 nm, 580-780 nm, 600-780 nm, 600-700 nm, 600-650 nm, 700-2500 nm, 700-800 nm, 800-2500 nm, 800-1300 nm.

An SPD for therapeutic applications may be defined by a target value of the relative power in the SPD within ranges, including the ranges above. For instance, the SPD may have about 10% of its power within a range 370-400 nm, 20% in the range 400-430 nm, less than 5% in the range 430-500 nm, and about 60% in the range 500-780 nm. Variations of plus/minus 5% or 2% around such prescribed values may be understood as typical tolerances for lighting applications.

Favoring one wavelength over another can be achieved in different ways. For example, in one embodiment, the preferred wavelength(s) is (are) amplified relative to the other wavelengths. For example, it may be a light bulb, a luminaire, a fixture, or a display system (such as a TV, a screen, a smart phone, a tablet). In a simple embodiment, this can be achieved using a light source having blue and red light, or a spectrum with enriched blue or red light. It is also possible to use a light source having a high illuminance, or emitting bright flashes of light. However, the use of a light source having a fixed spectrum may not be ideal. For example, it is not easy to ensure that the light source is adapted to the condition of the subject, which may evolve over time. In addition, light sources with such “therapeutic spectra” may suffer from poor quality of light (including tinted chromaticity and poor color rendering), which makes their use unpleasant or unacceptable.

Alternatively, the lighting system may be capable of dynamically tuning an emitted spectrum. For example, the lighting system may comprise a variety of narrow-band direct emitters, which can be turned on or off—including solid-state lighting emitters such as LEDs and laser diodes. In one particular embodiment, the lighting system may comprise an LED-based light source, which contains several LEDs emitting different spectra—these may be direct-emitter LEDs (such as ultra-violet, near-ultra-violet, violet, blue, green, yellow, red, infrared LEDs) or phosphor-converted LEDs (such as violet or blue LEDs pumping various phosphor mixes), or a combination thereof. The emitted spectrum is tuned based on the results of the visual acuity test. Such lighting systems are known and disclosed for example in U.S. patent application Ser. No. 14/531,545 and U.S. Pat. No. 9,368,695, incorporated herein by reference.

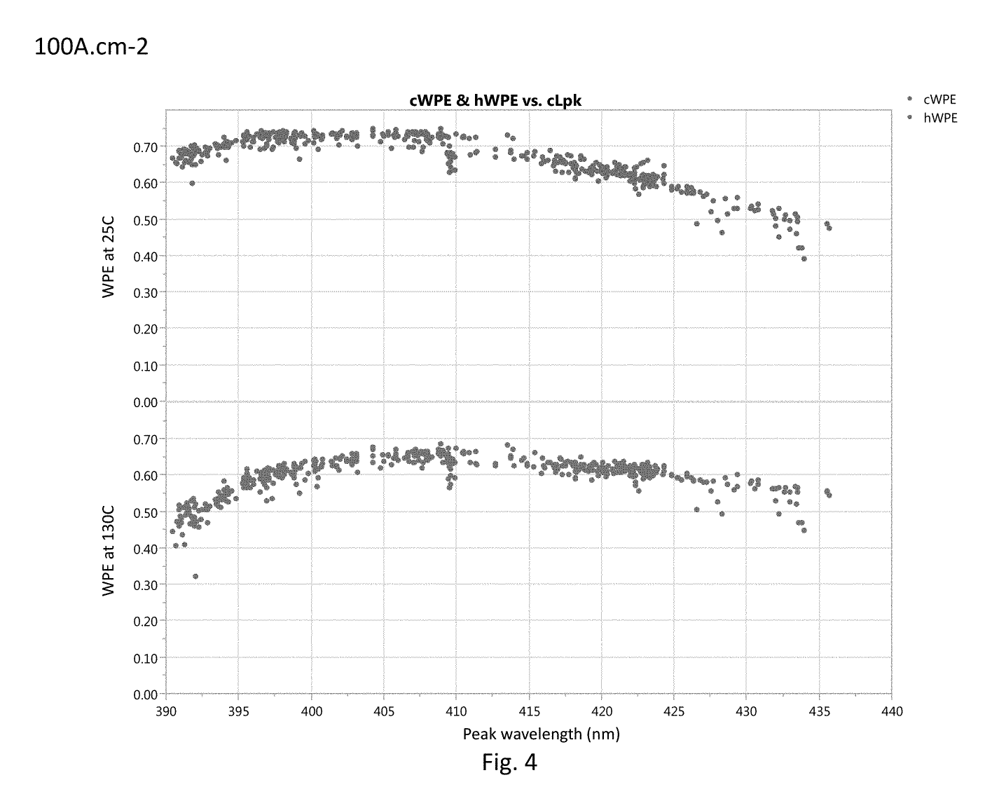

Light-emitting embodiments can be achieved with energy-efficient emitters such as solid-state emitters (lasers and laser diodes). In particular, for the wavelength range 370-530 nm, III-nitride solid-state emitters are known to be efficient. For example, referring to FIGS. 4-6, the efficiencies of various embodiments of the light sources used in connection with the present invention are considered.

FIG. 4 shows the absolute wall-plug efficiency (WPE) of III-Nitride LEDs in the wavelength range 390-440 nm, at a current density of 100 A·cm-2, at junction temperatures (equal to the base temperature here) of 25 C and 130 C. The performance is broadly maintained in the wavelength range 390-440 nm. A WPE above 50% is achieved in this range, at 130 C. The WPE slightly decreases at the edges of this range here, merely because the LED structures were optimized for the violet range. With further LED optimization, it is envisioned that similar performance can be achieved down to 370 nm (which is slightly above the bandgap of GaN) and up to the cyan or green range (i.e. up to 490 nm or 500 nm or 510 nm or 530 nm).

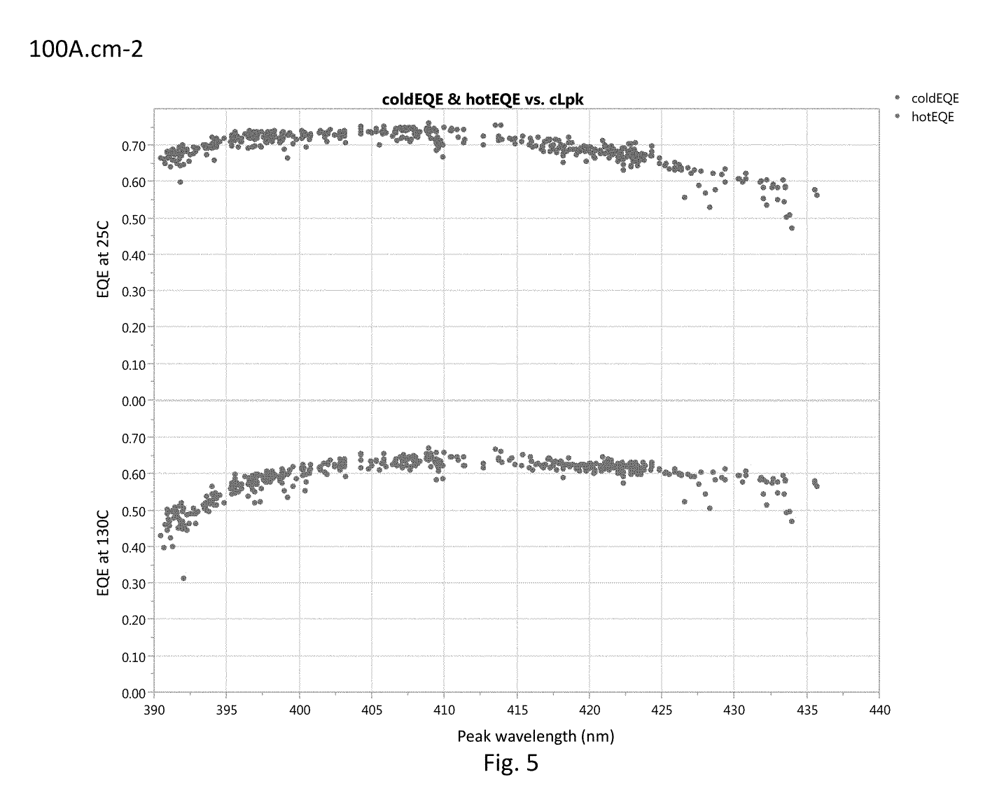

FIG. 5 shows the absolute external quantum efficiency (EQE) of III-Nitride LEDs in the wavelength range 390-440 nm, at a current density of 100 A·cm-2, at junction temperatures of 25 C and 130 C. The performance is broadly maintained in the wavelength range 390-440 nm. An EQE above 50% is achieved in this range, at 130 C. The same comments apply as for the EQE. Like the WPE results discussed above, the EQE slightly decreases at the edges of this range because the LED structures were optimized for the violet range. With further LED optimization, it is envisioned that similar EQE performance can be achieved down to 370 nm (which is slightly above the bandgap of GaN) and up to the cyan or green range (i.e. up to 490 nm or 500 nm or 510 nm or 530 nm).

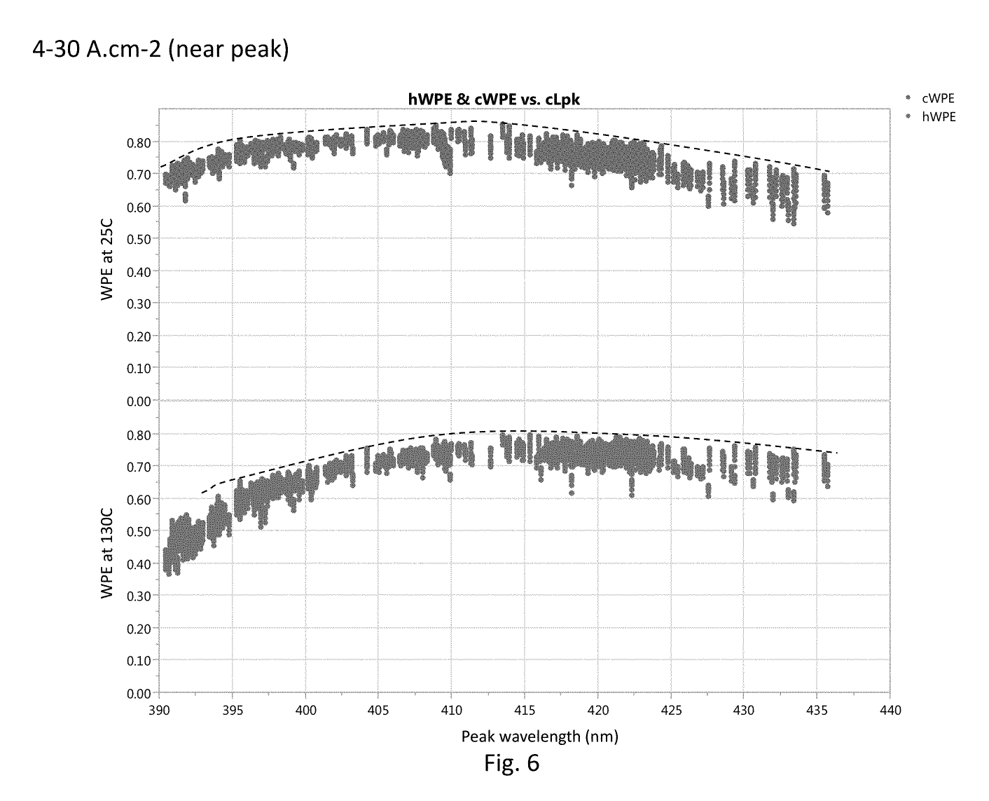

FIG. 6 shows the absolute WPE of III-Nitride LEDs in the wavelength range 390-440 nm, at various current densities in the range 4-30 A·cm-2, at junction temperatures of 25 C and 130 C. The performance is broadly maintained in the wavelength range 390-440 nm. A WPE above 70% is observed at 25 C. A WPE above 60% is observed at 130 C.

Likewise, it is known in the art that efficient emission can be obtained at other wavelengths. In the visible range, phosphor are available which can convert pump light (including violet or blue light) to green, yellow, orange, red or infrared light with a phosphor quantum yield better than 90%, and often better than 95%. Alternatively, direct emission from LEDs is efficient in the red and infrared range (for instance with the AlInGaP compound system, and other systems such as AlInGaAsP and derived compounds with dilute nitrides). Specifically, high-efficiency LEDs and laser diodes have been developed in the infrared range, for instance for telecommunication applications, including near 1300 nm (e.g., 1250-1350 nm) and near 1500 nm and 1550 nm (e.g., 1450-1600 nm). WPE for such LEDs may be above 50%, 60%, 70%, and even 80%, depending on the configuration and drive conditions.

In one embodiment, the system comprises a light monitor 106 for monitoring light the user is receiving to obtain exposure information, thereby allowing the does to be adjusted accordingly. This may be accomplished in a variety of different ways. For example, in one embodiment, the user carries or wears a light-detecting device for detecting and recording the amount of intensity and/or the wavelength spectrum the user is exposed to. The device may be a wearable, such as eye/sun glasses or a bracelet having a dedicated light detector, or a smart device such as, for example, a smartphone, tablet, or smartwatch. Alternatively, the system may comprise additional components disposed throughout the home/office for recording the user's exposure to light including the light's intensity and the spectral distribution. In one such embodiment, the system monitors the location of the user within the home such that the lights may be altered room-by-room depending on the user's location. Such embodiment may be preferable if multiple people within the family within the home are receiving different doses. Still other approaches for monitoring the user's exposure to light will be known to those of skill in the art in light of this disclosure.

In one embodiment, the evolution of the diagnosis is automatically communicated to the physician. For example, every six months, the user visits the physician and gets a full evaluation to confirm the assessment of the test system. The treatment is maintained over several months or years to reduce or eliminate the condition.

In one embodiment, the processor 101 is a freely programmable digital processor and is connected to additional components customary and necessary for its operation. To this end, memory 102 for data memory and/or programing is implemented in the integrated circuit configuration. In an exemplary embodiment, the data memory and the program memory are combined into a single memory configuration, but it is also possible to design them as individual memory devices. Apart from the possibility to connect the memory via a bus to the processor, additionally or alternatively the processor may be configured as a processor core with directly attached memory capacity. Alternatively, it should also be understood that the processor and associated computations may cloud-based as known in the art.

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, in the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

What is claimed is:

1. A method for treating an eye condition of a user using at least one light source, said at least one light source having an output which is adjustable in at least one of spectrum shape, intensity, or duration, said method comprising:
(a) obtaining information relating to an eye condition of said user;
(b) determining a dose of light to treat said eye condition, said dose including one or more of a particular spectrum shape, a particular intensity, or a particular duration; and
(c) causing said at least one light source to adjust said output to deliver said dose.

2. The method of claim 1, wherein said dose comprises each of said particular spectrum shape, said particular intensity, and said particular duration.

3. The method of claim 1, further comprising:
(d) monitoring said user's exposure to light to obtain exposure information and using said exposure information to revise said dose.

4. The method of claim 3, wherein step (d) is performed by a device carried by said user.

5. The method of claim 4, wherein said device is a laptop, tablet, smartphone, smartwatch, or a data-recording bracelet.

6. The method of claim 1, further comprising:
(d) reiterating steps (a)-(c) at least once.

7. The method of claim 1, wherein steps (a)-(c) are performed by a computer.

8. The method of claim 7, wherein step (a) comprises testing said user to determine said eye condition.

9. The method of claim 8, wherein said testing is performed by said computer, said computer having an imaging device to image said user's eye during said testing.

10. The method of claim 7, wherein said at least one lamp is a backlight for a display screen of said computer.

11. The method of claim 1, wherein the relative strength of at least one wavelength is adjustable relative to other wavelengths in said spectrum.

12. The method of claim 11, wherein step (b) comprises at least one of increasing said target relative strength of a wavelength between 300 nm and 500 nm to correct myopia, or increasing said target relative strength of a wavelength between 580 nm and 2500 nm to correct hyperopia.

13. The method of claim 1, wherein step (c) comprises wirelessly communicating with said at least one lamp to obtain said target relative strength.

14. The method of claim 1, wherein said at least one lamp is at least one of a desk lamp, a backlight for a display screen, or a plurality of room lamps.

15. The method of claim 14, wherein said plurality of room lamps illuminates one or more rooms of said user's residence.

16. A system for treating or preventing an eye condition of a user, comprising:
a digital processor operatively connected to at least one light source;
an input and output interface operatively connected to said digital processor for receiving and outing instructions and data to and from said digital processor;
memory operatively connected to said digital processor, said memory being configured with instructions for causing said processor to execute the following steps:
(a) obtaining information relating to said eye condition of said user;
(b) determining a dose of light to treat said eye condition, said dose including one or more of a particular spectrum shape, a particular intensity, or a particular duration; and
(c) causing said at least one light source to adjust said output to deliver said dose.

17. The system of claim 16, further comprising said at least one light source.

18. The system of claim 16, wherein said memory is further configured with instructions to determine said eye condition.

19. The system of claim 16, wherein said system comprises a light meter to monitor and record exposure information of at least one of intensity and spectrum over a certain duration.

20. The system of claim 19, wherein said memory also comprises instructions for using said exposure information to revise said dose.

21. The system of claim 16, wherein said dose is adjusted periodically.
